(19) United States
(12) Patent Application Publication
(10) Pub. No.: US 2002/0068869 A1
(43) Pub. Date: Jun. 6, 2002

(54) Drug delivery catheter with internal ultrasound receiver

(76) Inventors: Axel Brisken, Fremont, CA (US); Robert Zuk, Atherton, CA (US); John McKenzie, San Carlos, CA (US); Mark Cowen, Fremont, CA (US); Paul D. Corl, Palo Alto, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/893,341

(22) Filed: Jun. 26, 2001

Related U.S. Application Data
(60) Provisional application No. 60/214,600, filed on Jun. 27, 2000.

(51) Int. Cl. A61B 17/00

(52) U.S. Cl. 600/439; 601/2; 604/890.1

(57) ABSTRACT

Transducers and systems for applying ultrasonic energy to enhance drug delivery are illustrated.

### CROSS-REFERENCES TO RELATED APPLICATIONS

This application claims the benefit of prior provisional application No. 60/214,600, filed on Jun. 27, 2000, under 37 CFR §1.78(a)(3), the full disclosure of which is incorporated herein by reference.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### BACKGROUND OF THE INVENTION

The present invention provides a system for delivering therapeutic ultrasound to a patient and for targeting the ultrasound energy at a particular target region in the patient. In preferred aspects, the therapeutic ultrasound may be delivered for the purpose of gene therapy enhancement, or to inhibit intimal hyperplasia, however, any application of therapeutic ultrasound is included within the scope of the present invention.

In a preferred aspect, the present system is ideally suited for gene therapy enhancement of myocardial tissue. As such, the present invention extends the concept of external sonication of myocardial tissue (for gene therapy enhancement or inhibition of intimal hyperplasia) to applications involving therapeutic ultrasound therapy of peripheral and coronary arteries.

In further aspects of the invention, the present system is suited for sonication of any tissue or organ within the body, for the purpose of enhancing drug (including gene) delivery, for inhibiting hyperplastic tissue growth, for accelerating a healing response, or simply for providing guidance for any other form of therapeutic ultrasound exposure to the target tissue or organ.

In a preferred aspect, the present system comprises an external single element or an array (ie: multiple element) ultrasound transducer assembly with a catheter having an internal sensing transducer positioned thereon to assure treatment in a target region of tissue at a desired level.

As will be explained, the catheter comprises an internal ultrasound sensing transducer (ie: a hydrophone) mounted thereon which assists an operator in directing ultrasound energy (emitted from an external transducer) towards the internal sensor mounted on the catheter. The catheter can therefore be used for delivering drugs or genes (ie: gene therapy) to a target region of the patient (e.g. the myocardium or peripheral and coronary arteries). An external ultrasound transmitter can be used to deliver ultrasound to the target region (to increase gene transfection) and an ultrasound receiver transducer (ie: hydrophone) on the catheter can be used to direct the therapeutic ultrasound (from the external transducer) towards the catheter, (and therefore towards the target region into which the gene is delivered by the catheter). The catheter can be directed through any vessel in the body. Further, the catheter can be directed through any natural or man made lumen in the body.

### BRIEF SUMMARY OF THE INVENTION

As depicted in FIG. 1, the present invention comprises an internal catheter 10 and an external ultrasound source 12 which may comprise either a transducer or a transducer array.

The internal catheter further comprises a hydrophone 14 which acts as an internal receiver/sensor of the ultrasound delivered by external ultrasound source 12. Hydrophone 14 may be made from a very small piece of piezoelectric ceramic, in either rectangular or cylindrical shape. An advantage of using a cylindrical shape for hydrophone 14 (with the central longitudinal axis of the cylinder aligned parallel or collinear with the central longitudinal axis of the catheter) is that it will uniformly receive ultrasound from any radial direction.

In an exemplary cylindrical shaped aspect of the invention, hydrophone 14 is about 0.007 inches thick, about 0.030 inches in outer diameter, and about 0.020 inches long. An advantage of such cylindrical dimensions is that hydrophone 14 will have a uniform (isotropic) radiation pattern in both azimuth and elevation. The sensitivity need not be especially high. Optionally, catheter 10 may also have a lumen to traverse a guide wire, an injection/drug delivery needle and/or an angioplasty balloon, as sketched in FIG. 2. Hydroplane 14 may also be made from any other piezoelectrically active material such as PVDF or PVDF composite materials. Hydroplane 14 may also be made of non-piezoelectric materials such as micro-machined capacitor microphones.

The external transducer may preferably comprise a large aperture device as previously disclosed in U.S. patent application Ser. Nos. 09/255,290, filed Feb. 22, 1999; 09/3464,616, filed Jul. 29, 1999; and 09/435,095, filed Nov. 5th, 1999.

When operating external transducer 12, the acoustic beam is preferably focused at a depth equivalent to the target region (e.g. an artery or organ) intended for therapy. As sketched in FIG. 3, the focal zone will overlap the artery of interest. The output amplitude and emission timing would be according to the desired clinical application. (e.g.: desired depth, desired use, for example gene therapy or inhibition of intimal hyperplasia). In one embodiment, the ultrasound beam may sonicate only those tissues immediately surrounding the hydrophone catheter. In other embodiments, the external ultrasound transducer may sweep an acoustic beam over a larger volume of tissue in the proximity of the hydrophone catheter.

During operation, catheter 10 is advanced to the treatment zone. In optional aspects, catheter 10 is fitted with an angioplasty balloon (FIG. 2) such that it may be used to open an artery or assist in the placement of a stent. Catheter 10 is preferably positioned such that hydrophone 14 is placed into that portion of artery which requires treatment, (preferably under angiographic guidance). Subsequently, the larger external transducer 12 is positioned on the skin of the patient, with the appropriate ultrasonic coupling gel, and then moved or tilted until hydrophone 14 reports the maximum signal strength.

If the larger transducer is an array, it might also be positioned on the skin of the patient, with the appropriate coupling gel. The individual elements of the array would thence be polled to assess the time of acoustic propagation from the element to the hydrophone catheter tip. this timing data could thence be used to focus the external ultrasound transducer array on the target region or to allow the array to scan the beam, for example in a raster format, across a wider volume of tissue.

The hydrophone equipped catheter 10 preferably remains in place and measures the strength of the ultrasonic therapy after the therapy has been initiated. In an optional aspect of the invention, the hydrophone output is incorporated into a feedback loop, where the measured signal strength is used by the control system to drive the output power stages of the external transducer to deliver a desired dose of ultrasound. The operator observes the hydrophone output signal to keep the therapy beam on target. In the case of the feedback system, the operator seeks that position which results in the lowest output power of the control system as a means to keep the beam on target. Alternatively, with array transducers, the feedback system and the calibration periods might automatically keep the beam on the target organ.

In one exemplary embodiment (shown in FIG. 4), the control system would include an input channel from the hydrophone, an output channel to the therapy transducer, and a keyboard and display for operator control. The input channel may preferably comprise a simple AM receiver (amplifier, band pass filter, detector, low pass filter) and an A/D computer interface board. The control system preferably monitors the magnitude of the ultrasound signal on the artery. The output channel may preferably also comprise a digital I/O board to control a function generator and the output amplifier/driver gain.

In another exemplary embodiment, a transducer array (and accompanying driver) which can be scanned, or focussed, or both, such as previously disclosed in U.S. patent application Ser. No. 09/126,011 to externally deliver the therapy beam.

### PART II—PREFERRED EMBODIMENTS OF A MULTI-BEAM EXTERNAL TRANSDUCER SYSTEM 12

1. Overview:

In a preferred aspect, transducer assembly 12 comprises an easily implementable multi-beam transducer assembly for practical gene therapy applications. Multi-beam transducer assembly 12 is specifically well adapted for peripheral vascular ultrasound therapy.

Background:

Gene therapy experiments to date in the New Zealand White (NZW) rabbit model have clearly demonstrated the advantages of ultrasound therapy to enhance transfection rates of plasmid DNA expressing VEGF. In human studies, it has been determined that the injection bolus of DNA spreads across typically 5 cm of muscle tissue parallel to the muscle fibers and 2 cm perpendicular to the fibers. It has further been demonstrated that DNA degrades rapidly in the blood stream, by typically a factor of two in six minutes. Consequently, an effective transducer system will need to sonicate a large volume of muscle tissue in a rapid manner to achieve full potency of the treatment.

A problem with many existing transducer systems is that the beam width is so narrow (typically, on the order of 1 cm) that multiple sonications are needed to achieve coverage, but then time becomes a limiting factor.

To date, most medical ultrasound has been directed to imaging, where the smallest beam cross sectional area is preferred. More recent developments in hyperthermia and/or acoustic ablation have featured focussed ultrasound devices, in a step and repeat manner. To date, however, there has been no multi transducer device adapted for broad, wide beam width sonication coverage of the human body.

### BRIEF DESCRIPTION OF THE DRAWINGS

FIGS. 1-4 illustrate various systems according to the present invention.

FIGS. 4B illustrates operational parameters.

FIG. 5 illustrates circutory for use in the systems of the present invention.

FIGS. 6-9 illustrate an exemplory ultrasonic application.

FIGS. 10 and 11 illustrates energy distribution patterns.

FIGS. 12B-12C are erengy distribution charts.

FIGS. 13-15 illustrate different transducer patterns.

FIG. 16 illustrates an energy distribution pattern.

FIG. 17 illustrates control circuitry.

FIG. 18 illustrates use of the system in treating a patient.

FIGS. 19-17 illustrate frequency patterns.

FIGS. 29-29 illustrate multiplexing circuitry.

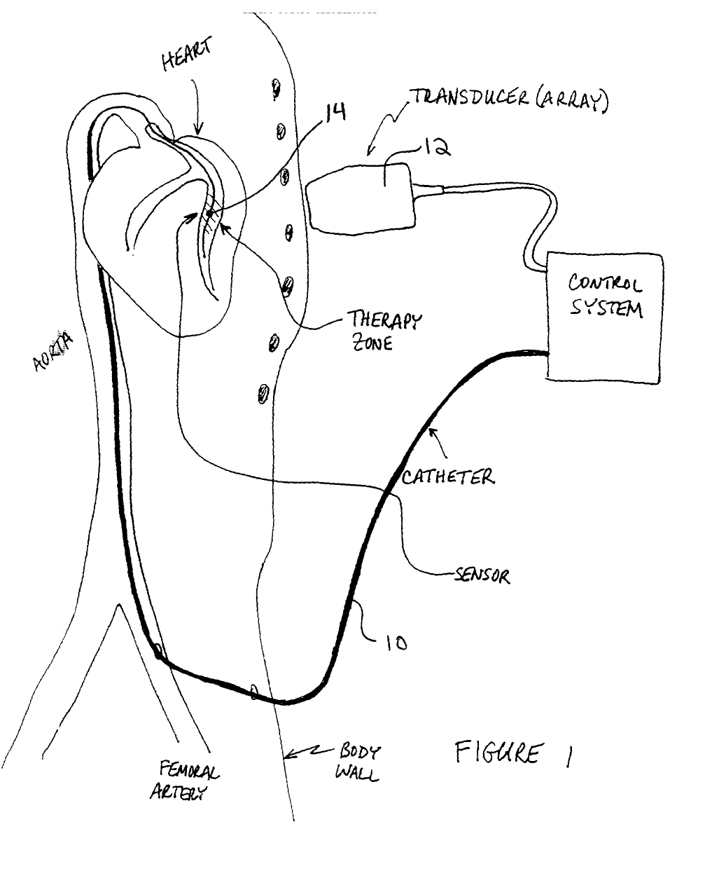

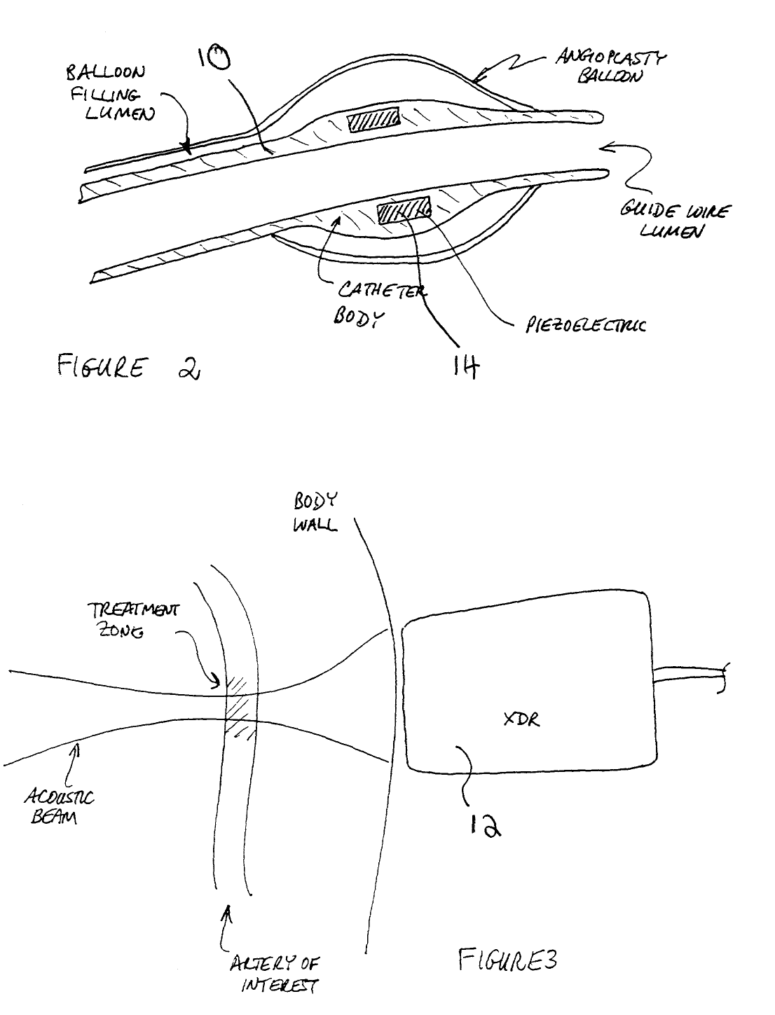

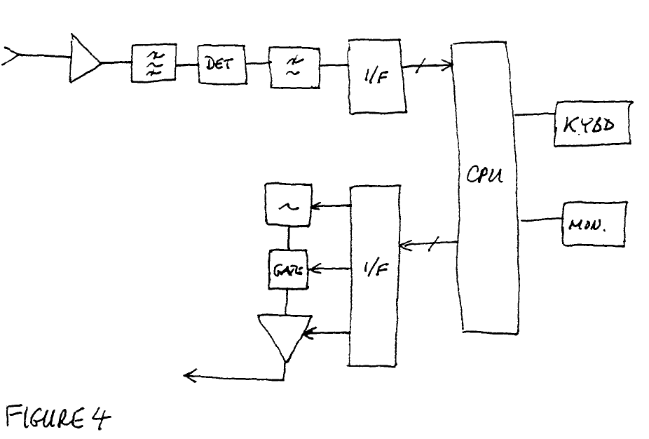

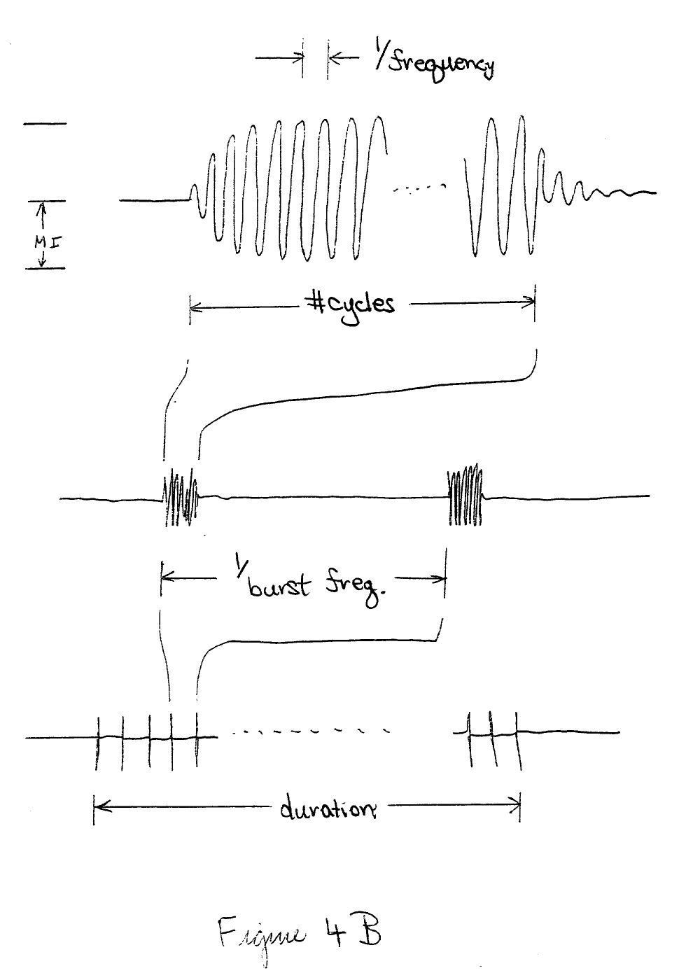

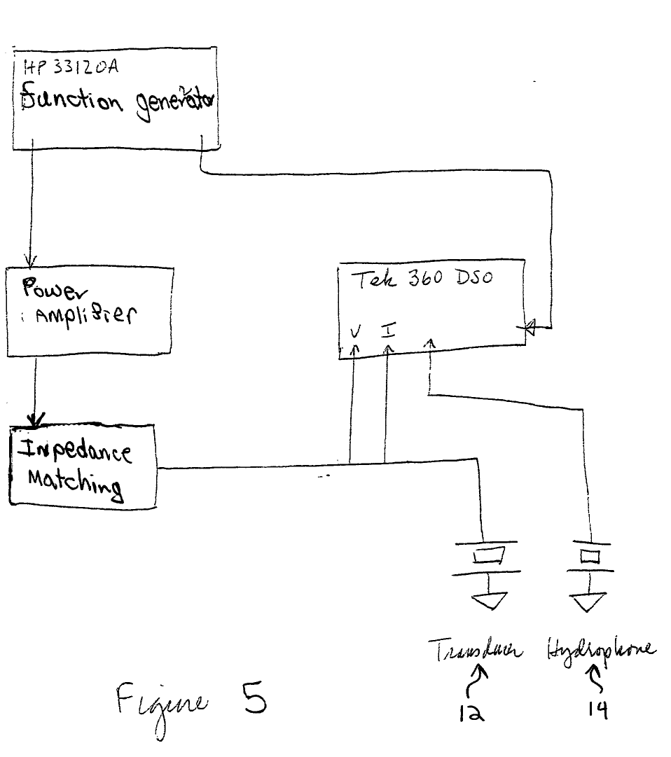

### DETAILED DESCRIPTION OF THE INVENTION

1. Overview:

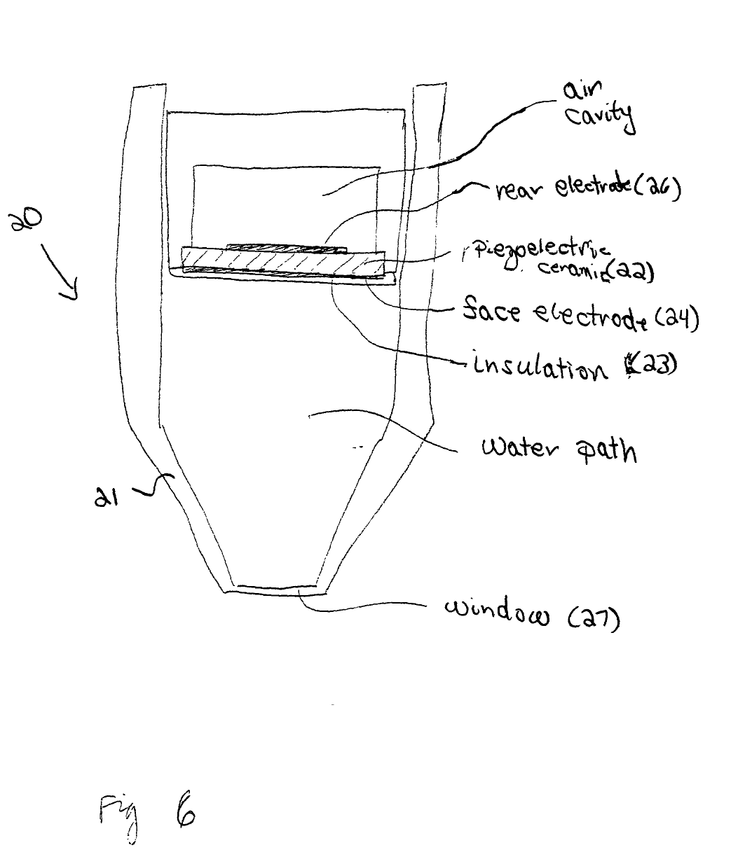

In accordance with exemplary embodiments of the present invention, repeatable results have been achieved in the NZW rabbit model with burst mode ultrasound, where the emission waveform is defined according to the sketch of FIG. 4B, and with the ultrasonic parameters which we define as the standard parameters (SP), as follows:

STANDARD PARAMETERS

| | Preferred | More Preferred | Exemplary |
|---|---|---|---|
| | Range | Range | Amount |
| Center frequency (MHz) | 0.2 to 5.0 | 0.3 to 3.0 | 0.947 |
| Amplitude (MI) | 0.2 to 10.0 | 0.5 to 5.0 | 1.8 |
| Burst mode cycles | 1 to 1000 | 2 to 5000 | 30 |
| Burst mode repetition | 0.1 to 100 | 0.5 to 20 | 1.893 |
| frequency | | | |
| Duty cycle (%) | 0.1 to 50 | 1 to 20 | 6.0 |
| Exposure time (sec) | 10 to 1800 | 30 to 900 | 60.0 |
| Beam width at −6 dB (mm) | 2 to 50 | 5 to 25 | 10.0 |

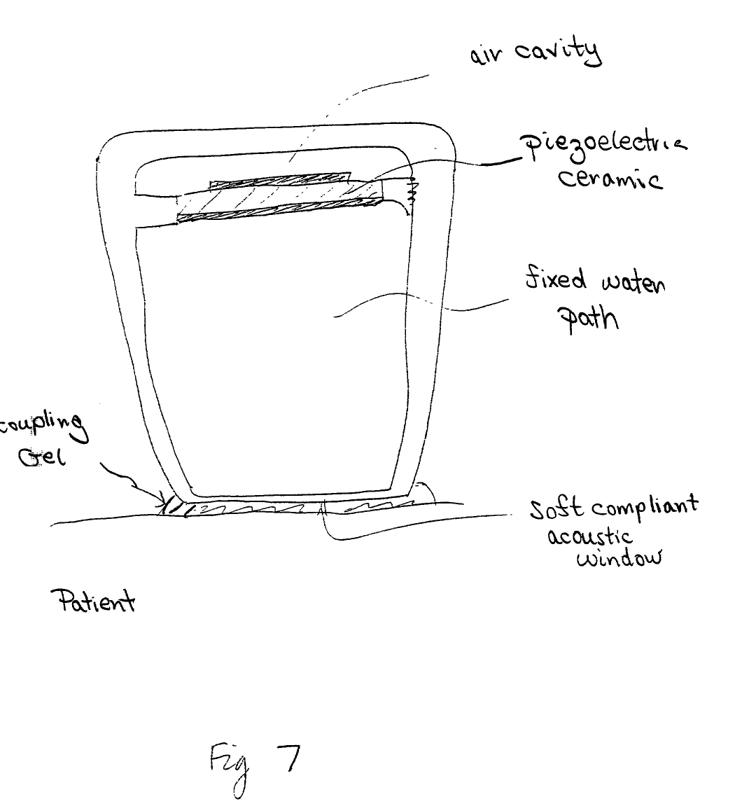

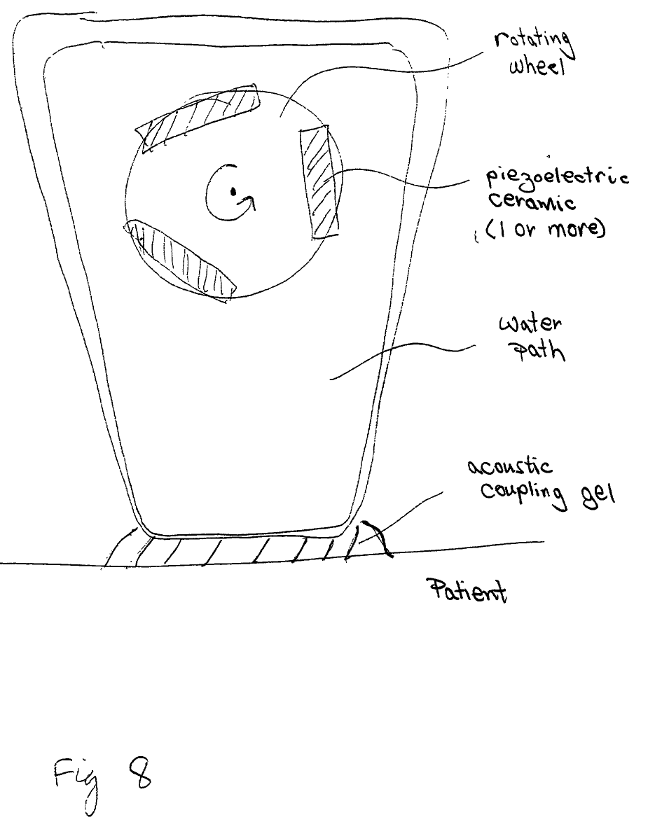

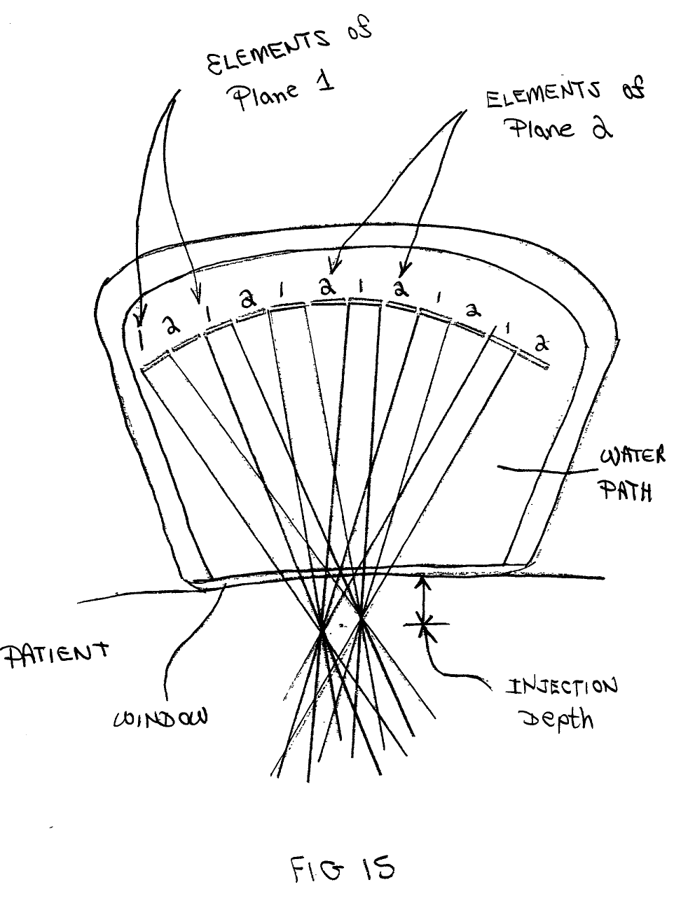

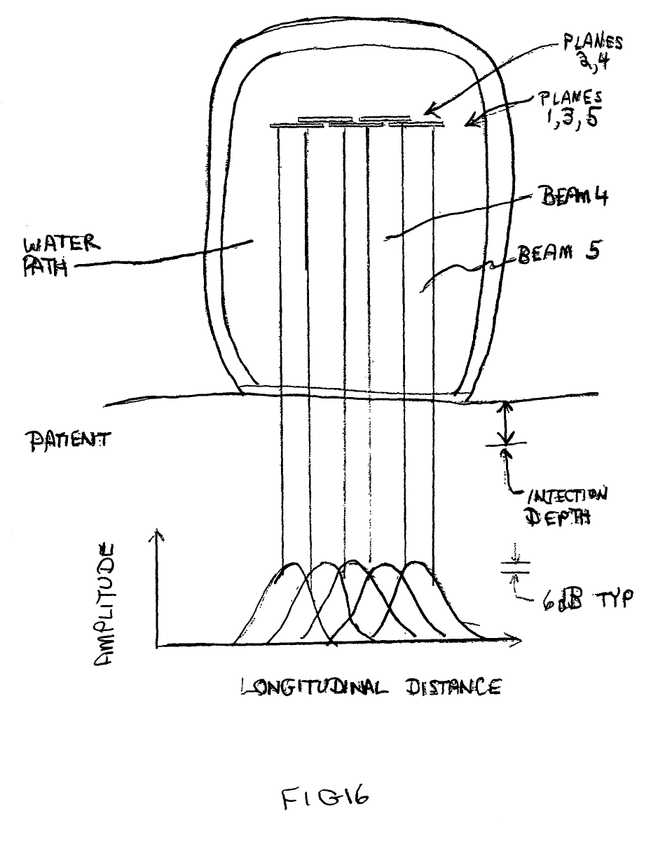

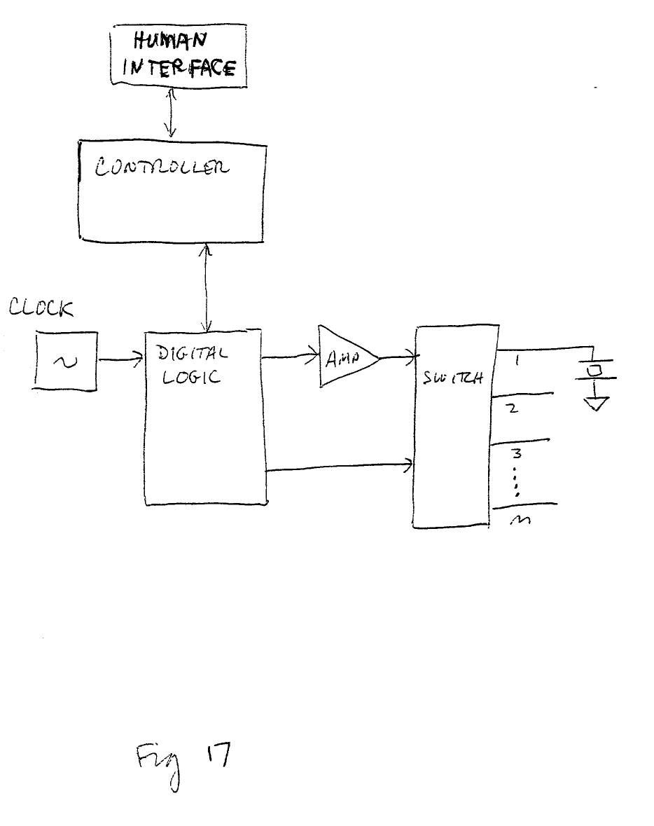

The center frequency is the transmission frequency as defined, for example, by the signal generator of the equipment configuration sketched in FIG. 5. The transducer amplitude is defined by the output of the power amplifier as measured by a broad band PVDF hydrophone, on beam center, at that distance in front of the transducer corresponding to the injection depth of the plasmid DNA. Mechanical Index (MI) is defined as the peak negative pressure of the burst waveform (in units of MPa) divided by the center frequency (in units of MHz). System performance is monitored by observing the voltage and current on the line to the transducer during all sonication periods (and during calibration). The number of cycles during the burst and the burst repetition rate are defined by the signal generator. The duty cycle is defined as that portion of time during which the power amplifier is energized. FIG. 12A depicts a typical modeled axial beam profile along the central axis of the transducer from the transducer face outward. The vertical axis reflects signal strength in dB while the horizontal axis reflects axial distance. FIG. 12B depicts typical modelled lateral beam profiles at and around the injection depth of the plasmid DNA, typically 6 mm (+/−) from the transducer face. The vertical axis reflects signal strength in dB while the horizontal axis reflects lateral distance, with beam center at the left side of the profile. The beam width is defined for the −6 dB level with respect to the peak amplitude. Beam profiles are measured with the transducer face down, just touching the surface of water, above a broad band hydrophone. This measurement configuration most ideally matches the operational configuration, with acoustic coupling gel between the transducer face and the animal subject. Typical beam profiles in both the axial and lateral directions show a small ripple pattern in the response, as compared to the modelled typical sinx/x response. This is due to acoustic energy which couples to the conical housing of the transducer assembly and causes constructive/destructive interference with the energy propagating directly in an unobstructed manner through the front window.

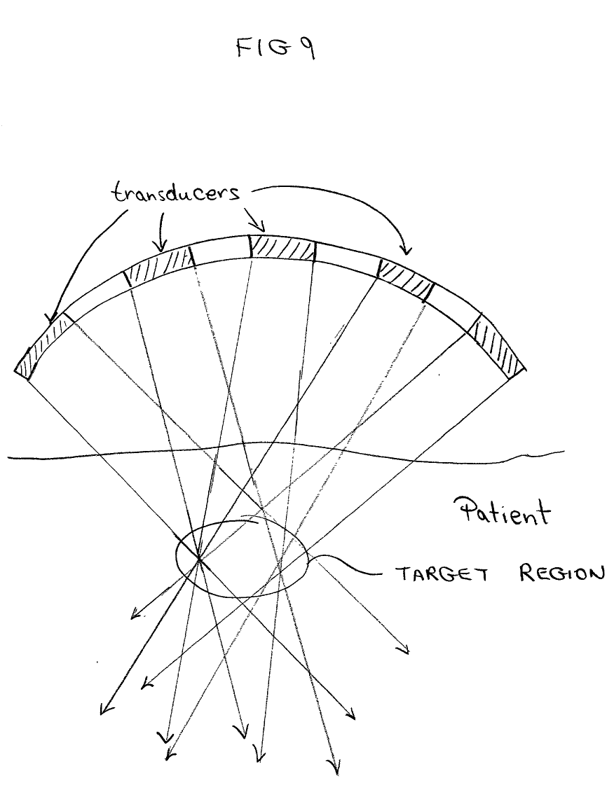

FIG. 6 depicts the transducer configuration used in animal experiments to date. The system assembly 20 comprises a 1.5 inch diameter piece of PZT-8 piezoelectric ceramic 22, approximately 0.080 inches thick. A first electrode 24 connected to a system ground (not shown) covers the entire front face of ceramic 22. This in turn is covered by several thin layers of acrylic 23 for electrical isolation. A second electrode 26 connected to the output of the system power amplifier (not shown) and impedance matching circuit (not shown) covers a 1.0 inch diameter area in the center of the back surface of piezoelectric ceramic 22. Ceramic 22 is air backed. Ceramic 22 is mounted in a polycarbonate housing 21 with epoxy on the edges. Ceramic transducer 28 may preferably be mounted in polycarbonate housing 21 with a tapered cone and a 0.005 inch thick polycarbonate acoustic window 27. During operation, assembly 20 is filled with bubble free water. The water path distance is set to approximately 100 mm between the face of transducer 22 and the front surface window 27. The ultrasonic signal from the transducer peaks at approximately 106 mm from the face of ceramic 22, or 6 mm from the polycarbonate window 27.

2. Considerations for human subject clinical implementation:

A starting point for human clinical studies will be the standard ultrasound conditions (SP) detailed above. In accordance with the present invention, system parameters (including, but not limited to, the frequency, the amplitude, the burst length, and the burst repetition period) can be adjusted to increase the efficacy of any particular plasmid DNA at any particular application site.

Previous studies have shown that the injected bolus of plasmid DNA spreads quickly within the muscle mass over a distance of typically 5 cm parallel to the muscle fibers and 2 cm perpendicular to the fibers. This suggests that the sonication area needs to be approximately 10 cm² with a depth of field of approximately 2 cm. Furthermore, studies have shown that the plasmid DNA is naturally degraded to half potency in the blood stream within approximately 6 minutes. This suggests that all of the sonication preferably needs to be completed at least within that time frame.

The Thermal Index (TIₜ) for normal vascularized tissue may be simply expressed as the time averaged acoustic power at the source Wₒ (in units of mW), multiplied by the center frequency (in units of MHz), and divided by 210. When acoustic energy strikes bone, however, the Thermal Index (Ti_{b}) may be simply expressed as the time averaged acoustic power at the source Wₒ, divided by 40, and divided by the equivalent aperture diameter D_{eq} (in units of cm).

In terms of the conditions used for current experiments, these simplified expressions would suggest a tissue temperature increase of 30 Centigrade degrees and a tissue/bone temperature increase of 150 degrees. However, neither of these temperature excursions are realized, in part due to the fact that ultrasound is applied for only a short period of time. The above simplified expressions for Thermal Index assume a long term, continuous application of ultrasound. As heat builds up in the body, however, greater thermal conduction will take place. Indeed, using an absorption coefficient of 0.3 dB/cm/MHz, one would expect to see only a six degree temperature rise in soft tissue in one minute, assuming no loss mechanisms. Thermal conduction alone will reduce this value, and circulatory mechanisms would also remove more heat. Accordingly, temperature excursions of less than four degrees have been measured in animals to date, with the standard ultrasound conditions.

The calculations of Thermal Index, however, serve an important purpose, in that they raise an awareness of the need not to continuously project high levels of ultrasound against bone. Absorption of ultrasound on the bone will increase the local temperature and may cause unwanted biological effects. For example, reducing the ultrasound exposure time against the bone by a factor of five compared to the sonication time would suggest an equal distribution of heat in the body between tissue and bone.

In accordance with the present invention, (and the standard parameters set forth above), the following “FIRST ORDER SONICATION CONDITIONS” (which correspond to the above “Exemplary Amounts” in the “STANDARD PARAMETERS” table above) are anticipated.

| | |
|---|---|
| Center frequency (MHz) | 1 |
| Amplitude (MI) | 1.8 |
| Burst mode cycles | 30 |
| Burst mode repetition frequency (kHz) | 1.893 |
| Duty cycle (%) | 6.0 |
| Exposure time (sec) | 60 |
| Exposure volume | |
| Length parallel to muscle (cm) | 5 |
| Width perpendicular to muscle (cm) | 2 |
| Depth of field (cm) | 2 |
| Typical plasmid DNA injection depth (cm) | 1.5-3.0 |
| Acceptable temperature rise (tissue and bone) (TI) | <4 |

In clinical practice, plasmid DNA injections are typically placed in the large muscles of the leg. In the typical human subject, these injections are made in eight locations at depths of typically 2.5 cm. However, human subjects vary considerably due to variations in muscle mass. Ideally therefore, injections should not all be made at the same depth in all patients. Furthermore, the depth and amount of muscle mass vary from one location to another on the leg. Lastly, peripheral limb edema may cause massive swelling of the leg requiring significantly deeper injections. Consequently, the present invention provides systems with the ability to easily adjust the depth of focus, or the depth of greatest sonication intensity.

3. Overview of device design approaches:

The present invention provides a variety of systems which are adapted to achieve the sonication conditions set forth above. Four approaches are summarized in the table below:

HUMAN SUBJECT SONICATION OPTIONS

| DESIGN OPTION | ADVANTAGES |
|---|---|
| Single element | Simple design |
| manually scanned | Low development cost |
| device | Short development time |
| | Ease of use |
| Mechanical sector | Simple transducer element |
| | Moderate development cost |
| | Moderate development time |
| Multibeam probe | Simple design |
| | Moderate development cost |
| | Moderate development time |
| | Ease of use |
| 2 Dimensional | Elegant design |
| Phased | Complete electronic scanning control |
| Array | Cover total tissue volume with multiple beams |
| | Adjustable depth of field |

The first (Single element manually scanned device) approach may preferably comprise a single circular aperture with a water path, similar to the device used for the animal experiments. Exemplary embodiments of such systems are set forth in co-pending U.S. patent application Ser. Nos. 09/255,290, filed Feb. 22, 1999; 09/3464,616, filed Jul. 29, 1999; and 09/435,095, filed Nov. 5th, 1999.

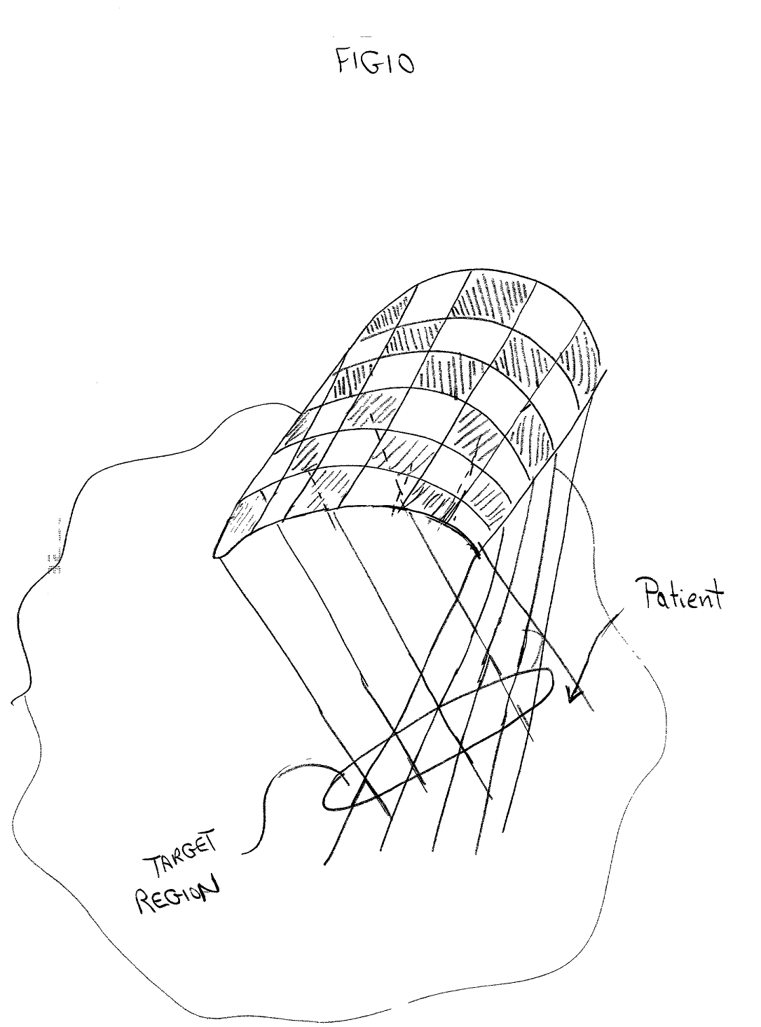

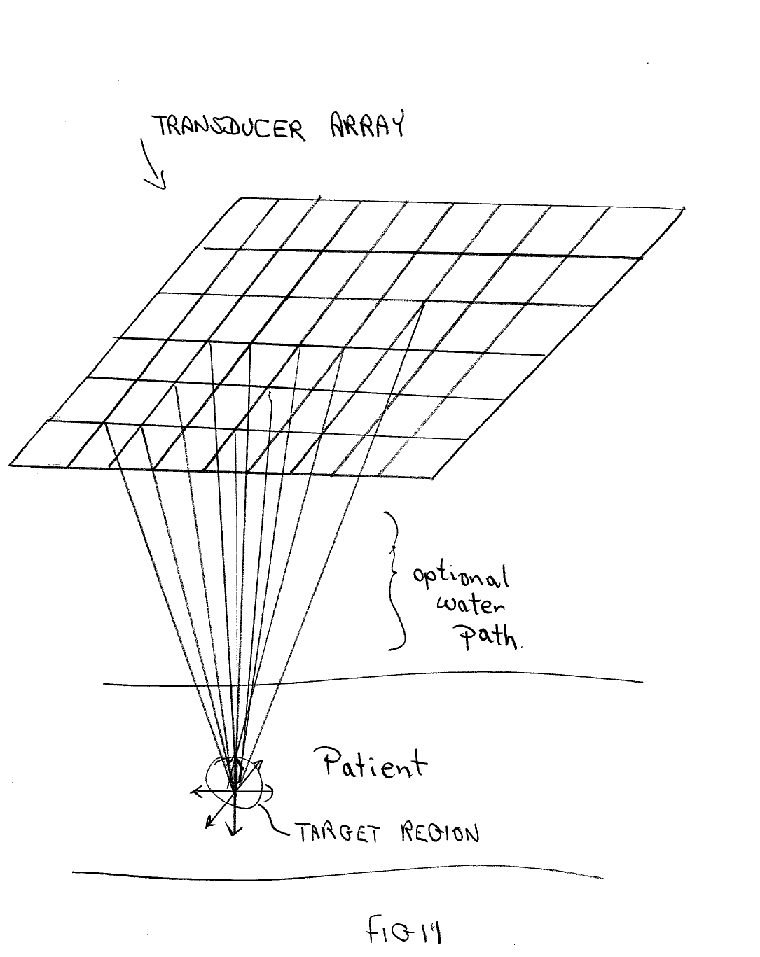

As illustrated in FIG. 7, the device would feature a transducer with a water path applicator. The patient contact surface of the water path is preferably sufficiently large so as to not interfere with the beam (for those beam components greater than about −12 dB from peak amplitude). The transducer in such assembly may preferably comprise a single flat unfocussed plate of piezoelectric ceramic for operation at the natural focus, or a mechanically focused plate, or an annular array assembly. This device will be described further below.

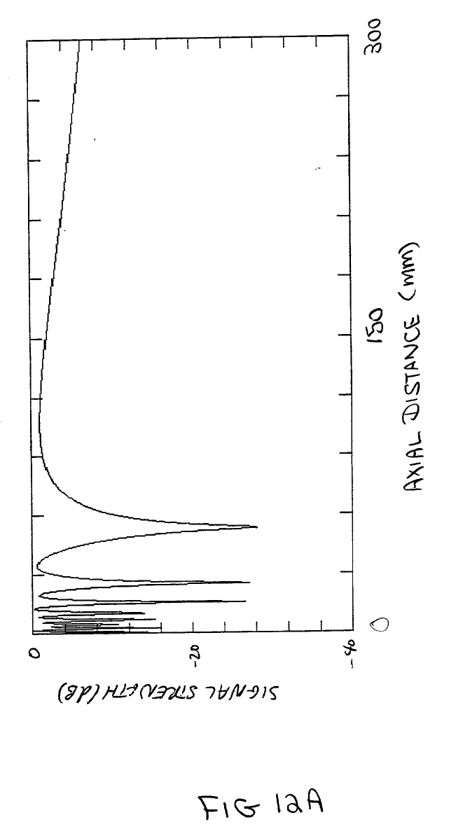

The second (Mechanical sector) approach comprises using one or more of the above transducers mounted onto a rotating wheel in a mechanical assembly, as sketched in FIG. 8.

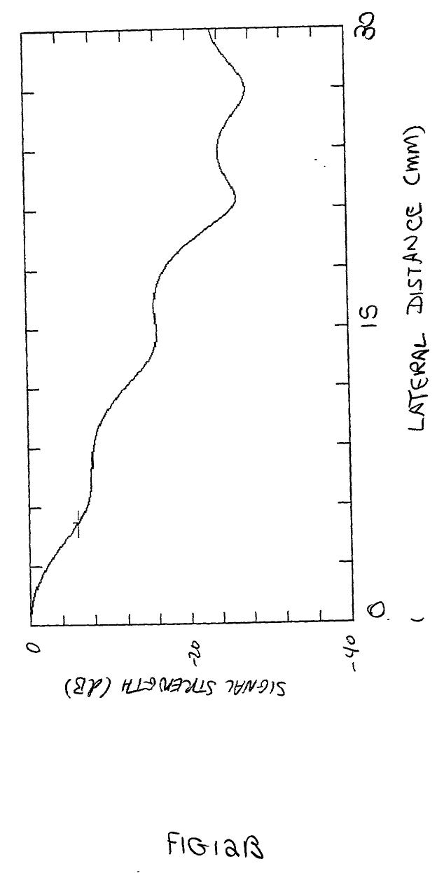

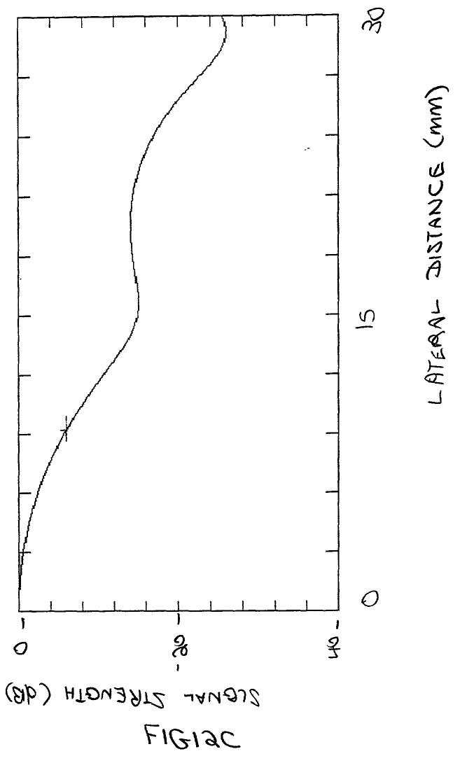

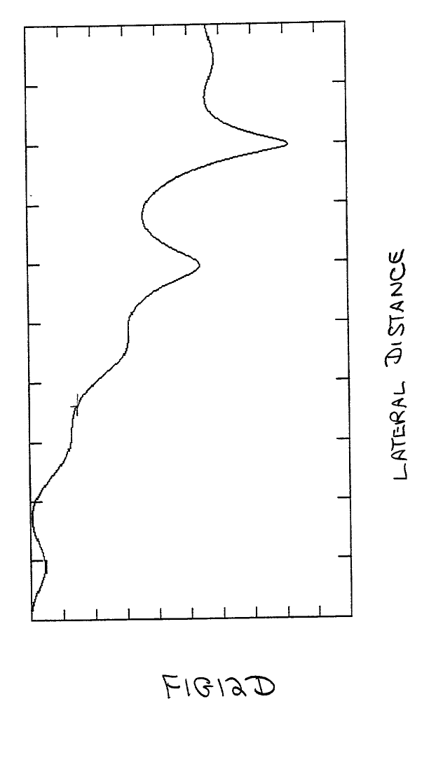

The third (Multibeam probe) approach comprises using multiple single element transducers from above arrayed into a fixed grid, on a cylindrically curved surface, for multi-beam treatment of the entire volume of injectate, as sketched in the end view of FIG. 9 and the perspective view of FIG. 10. By having continuous interleaving operation of different subsets of transducers, any and all sections of sonicated tissue will experience the requisite pulse repetition rate and amplitude, but from different directions, such that direct sonication of bone is substantially limited. This device will be described further below.

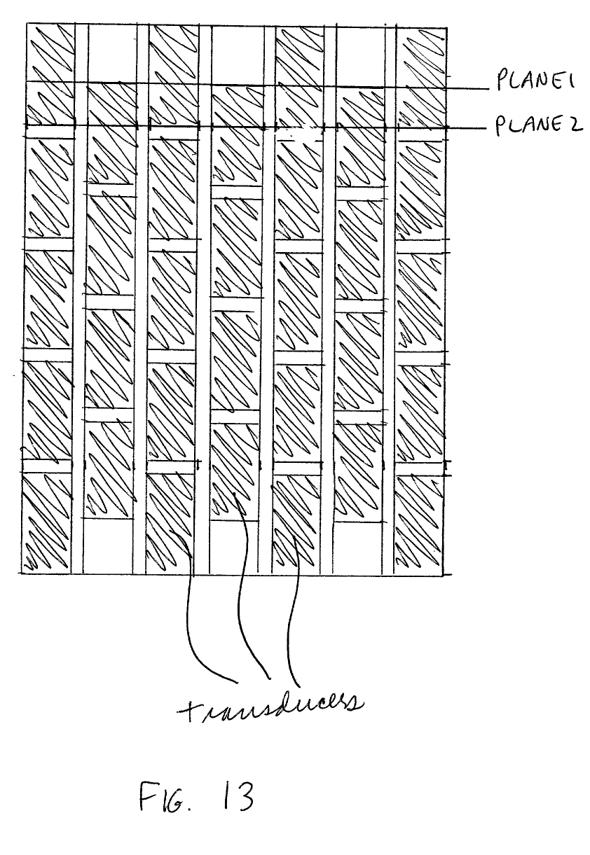

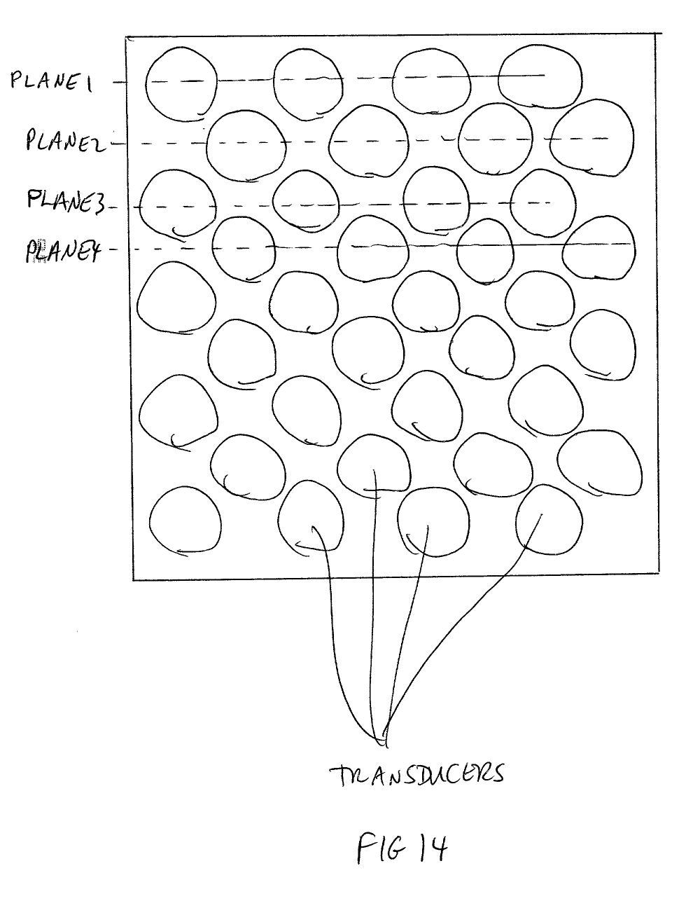

A fourth (2 Dimensional phased array) approach preferably comprises using a two dimensional phased array as sketched in FIG. 11. This array may preferably be implemented with a water path buffer disposed between its radiation surface and the target tissues in the subject patient. The beam of such a phased array may be in continuous operation, although any section of designated tissue would preferably see only the requisite short duty cycle of exposure. To limit bone exposure, the system would preferably have sharp focal zones (with rapidly divergent beams beyond these focal zones). At any one time, any or all of the array elements might be used for sonicating a tissue sample. Multiple beams directed toward separate tissue samples are also contemplated.

An advantage of the Multibeam Probe (FIGS. 9 and 10) and 2 Dimensional phased array (FIG. 11) approaches of the present system are that they provide an adjustable depth of field. In the case of a multi-beam probe, the entire cylindrical curved surface may be manually adjusted to place the junction of the intersecting beams at any required depth in the body. In the case of the 2 dimensional phased array, the timing of transient signals to array elements may be adapted to focus the array at any selected tissue depth. An advantage of the phased array approach is that this fixed depth may be adjusted during therapy to sonicate variable depths of tissue.

4. Exemplary Embodiments of the Four Above Approaches to External Transducer System Design:

a. Single Transducer—manually or mechanically scanned device:

In a simple aspect of the present invention, the single element device comprises a flat plate of piezoelectric ceramic and a water path, as sketched in FIG. 7. The modeled axial beam profile from such a device (1 MHz, 25.4 mm diameter) is sketched in FIG. 12A, wherein the vertical axis depicts signal strength in dB and the horizontal axis reflects the distance from the transducer face outward. The modeled device features a large depth of field, with a natural focal zone which is set to peak at the desired depth in the patient's body (typically about 107 mm from the device surface). The modeled lateral beam profile from such a device is the simple sinx/x pattern, as shown in FIG. 12B, with a 10.4 mm −6 dB beam width. The vertical axis again depicts signal strength in dB while the horizontal axis reflects distance from beam center laterally outward.

As can be seen in FIG. 12A the present device can cover the 2 cm depth of a typical injectate volume. To cover the 2×5 cm cross sectional area of the injectate volume, this device is preferably moved to different locations on the patient's body.

In a preferred aspect, the water path is preferably extended. FIG. 12C depicts the modeled lateral beam profile from twice the natural focal distance, displaying a −6 dB beam, having a beam width of 18.5 mm. Alternatively, short of the natural focal zone, at approximately 70 mm, there exists a region approximately 15 to 20 mm deep wherein the beam width runs 15 to 20 mm wide within a 6 dB amplitude variation, albeit perhaps 8 dB down in amplitude. FIG. 12D depicts the modeled lateral beam profile at 70 mm range. These improvements in beam width substantially reduce the number of sonications required.

In optional preferred aspects, the beam width may be increased to even larger dimensions (to ensure that a reasonably sized circular aperture would be able to cover the entire volume of injectate diffusion with one sonication). The beam width may preferably be increased to these larger dimensions by reducing the aperture of the device. Operating with a larger aperture and an extended water path standoff will also increase the width of the lateral beam profile.

In accordance with the present invention, an annular array is provided to effect dramatic increases in lateral beam width. In an exemplary embodiment of the invention, a central disc with one annulus, for example, operating at 1 MHz where the one inch central disc is driven at the normal drive level and the one annulus with an outer diameter of 1.42 inches is driven at 0.5 times the normal drive level yields a lateral beam with of approximately 24 mm.

A significant advantage of the single element device is its simplicity and the accompanying simplicity of the driving electronics. As sketched in FIG. 5, the driving electronics comprises a simple signal generator, a power amplifier, and an impedance matching circuit. The duration of exposure at any location can be timed manually.

b. Multibeam devices:

In accordance with various preferred aspects of the present invention, therapeutic ultrasound beams are typically directed into the human body from multiple different directions so as to achieve a high dose in the selected tissue volume but lower doses in the surrounding tissue mass. As illustrated in FIG. 9 and 10, the tissue containing the injectate (ie: the “target region”) is exposed to ultrasound from different directions, each being generally perpendicular to the target region. As can be seen in FIG. 10, the target region may preferably be elongated (in the direction in which the drug/gene diffuses after injection.

In a preferred aspect, each individual transducer of the transducer assembly preferably transmits for only a portion of the duty cycle, such that the accumulated illumination from all transducers in each plane amounts to the desired net duty cycle in a target region of the tissue. The transducers may be staggered such that the direction from which various ultrasound beams are directed to the target region is continually changing. Alternatively, transducers which are directed in different directions may be energized at the same time provided that the beam profiles do not overlap so as to cause constructive or destructive interference. In this manner, the entire volume of tissue can be sonicated in one sonication period, at the desired duty cycle.

An advantage of directing ultrasound toward the target region through a variety of different pathways (as is accomplished when directing the ultrasound from a plurality of different transducers in a transducer array) is that the effects generated by the ultrasound are large enough to be therapeutic in the target region itself, but are sub-therapeutic elsewhere. As such the potential for heating at the tissue/bone interface is reduced.

In optional aspects, the entire assembly of transducers comprises many “planes” of transducers, as sketched in FIG. 10, (for comparison, FIG. 9 shows one “plane” of transducers) where the number of planes is such that the entire length of the target region (ie: region of diffused injectate) within the tissue may be exposed with ultrasound. In other optional aspects, transducers of any one plane will be partially interleaved with transducers of the next plane so as to provide a more uniform illumination of tissue, as will be explained.

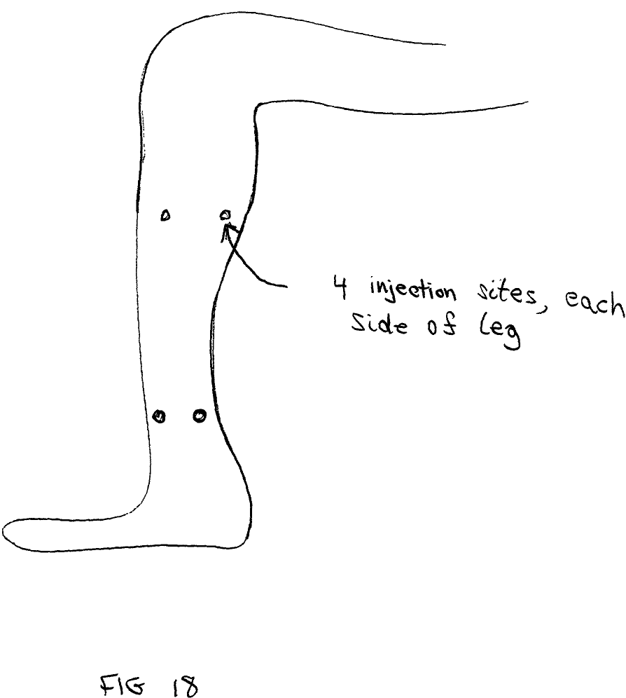

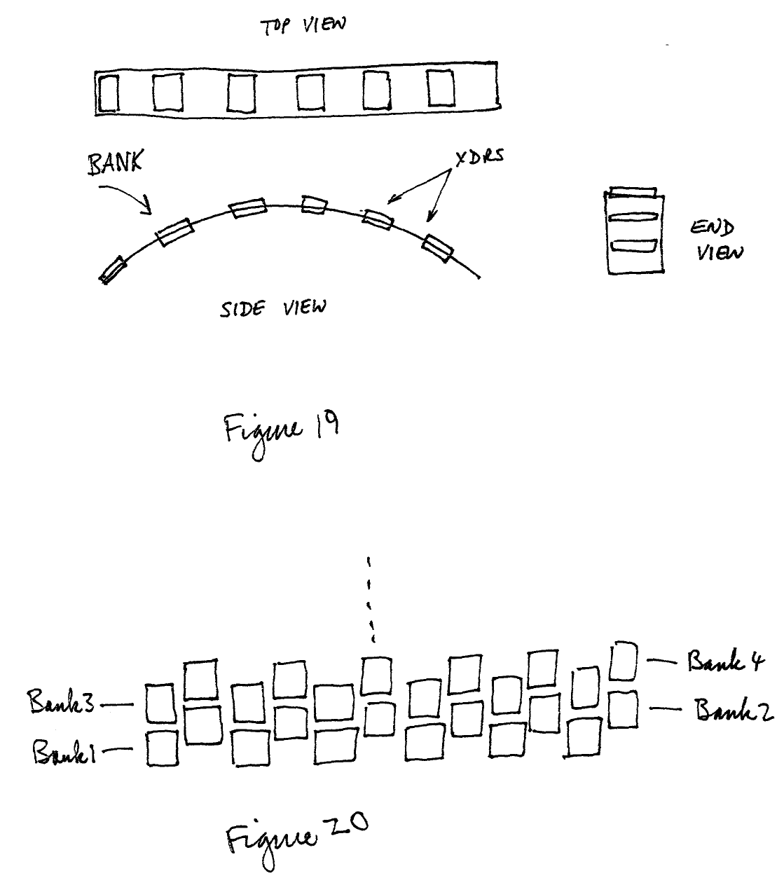

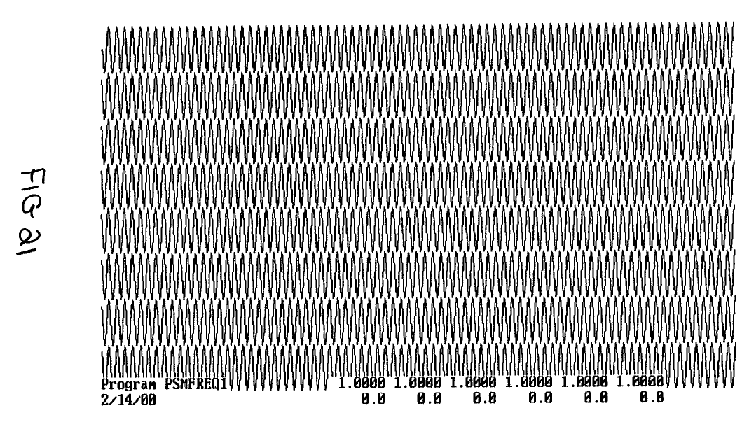

The subset of individual transducer elements in any one plane may be spaced apart by any fixed amount. In practice, the spacing will be dictated by the amount of interleaving required such that a sufficiently long section of tissue can be scanned with a net uniform field. FIG. 13 depicts the unfolded cylindrical surface of the transducer array of FIG. 10 (with rectangular shaped transducer elements). FIG. 14 depicts a comparable transducer array with circular transducers with a maximum packing density, although elliptical elements may also be used. In all cases, the transducers are preferably spaced apart only by a small margin to provide for the mechanical structure of the mounting assembly. Alternate planes of transducers are preferably interlaced to the maximum extent. FIG. 13 depicts two interleaving planes. It is to be understood, however, that depending on the actual size of transducer elements, a greater number of interleaving planes can be used.

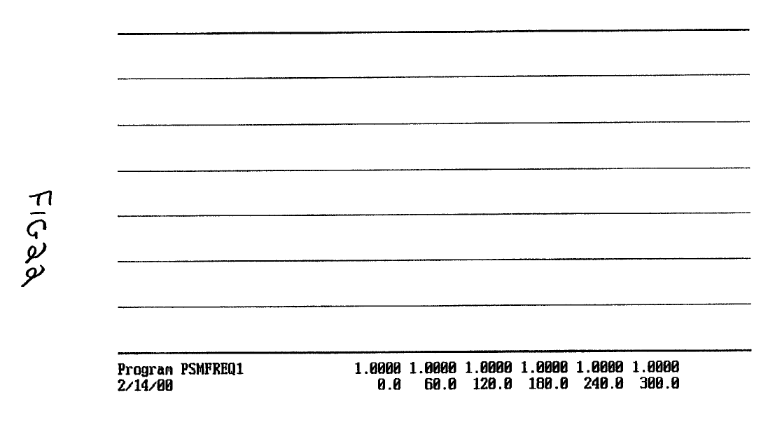

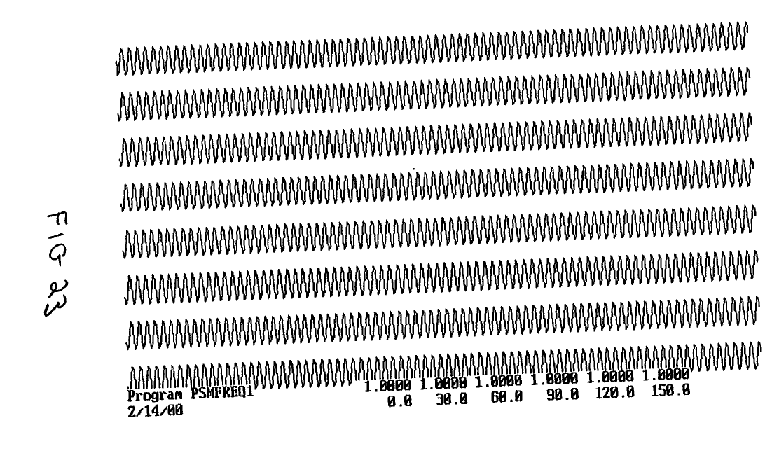

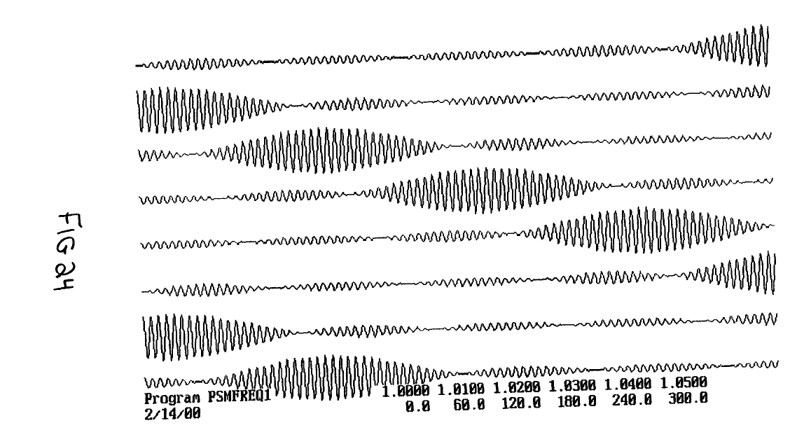

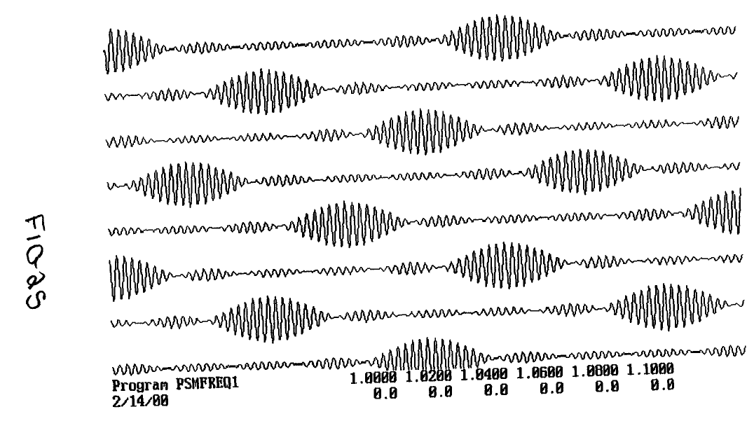

In an exemplary embodiment of the present system, as illustrated in FIG. 13, a 1 MHz assembly of rectangular transducer elements, arrayed on the cylindrical surface with a radius of 162 mm, with each element 16 by 26 mm wide, and with 2 mm gaps between elements is provided. With six elements per plane, the arc of the planes extends over 212 mm, or 75 degrees of arc, as illustrated in FIG. 15. The ultrasonic beam from each of the transducers of the plane is also shown. In the longitudinal direction, five interleaved planes cover a distance of 82 mm. Each element will have an in-plane −6 dB beam width at 1 MHz of 19.3 mm and a cross-plane beam width of 15.3 mm. The distance between the outside edges of the −6 dB profiles is 71.3 mm. FIG. 16 illustrates the parallel planes of transducers and the net beam profile in the longitudinal direction. In the axial direction, the depth of field of each transducer element exceeds 20 mm. Given the 19.3 mm individual element beam width and the net 71.3 mm longitudinal coverage area, it is seen that the assembly is capable of easily covering the entire volume of injectate diffusion with one placement. Furthermore, assuming a need for a 6 percent duty cycle, each element per plane needs to operate for only one percent of the time. Consequently, there is no need to overlap operation of any of the devices in the assembly, necessitating the need for a simple switched driver (one variation of which is sketched in FIG. 17).

The present multibeam device, with five planes of six devices each (by way of example) will easily sonicate a large tissue volume with a moderate duty cycle, with one placement of the assembly.

Current Animal Studies:

Current animal studies have been performed under the following parameters, (however, the present invention is not limited to applications under these parameters):

Burst Length: 30 cycle burst lengths, typically with a maximum burst amplitude within 5 to 6 cycles. (However, 5 to 6 cycle burst lengths will also result in improved transfection rates of DNA, especially when such improved transfection rates is the result of a mechanical effect).

Wavelength: current animal studies were conducted on the hind limb of NZW rabbits, where the maximum tissue thickness was on the order of 20 mm. A 30 cycle burst length, with a 1.5 mm wavelength was used.

Frequency: typically 947 kHz. Lower and higher frequencies (for example 0.5 and 2.0 MHz) are also contemplated.

Burst repetition rate: 6 percent duty cycle, at a burst rate of 1890 Hz.

Drive amplitude: typically 1.8 MI. (Higher exposure levels may result in improved transfection).

Total ultrasound exposure: one minute.

Human subject clinical trials:

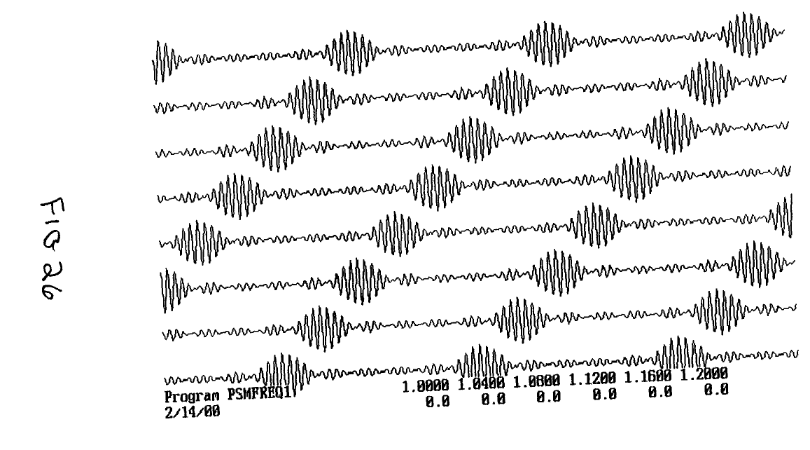

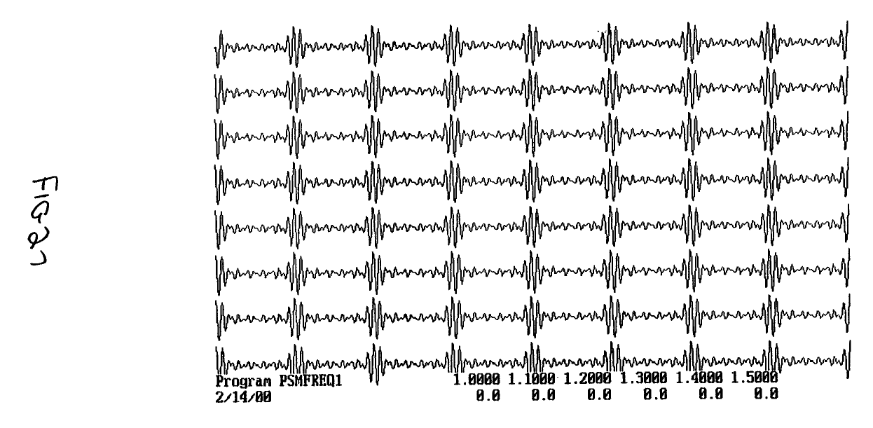

Currently, patients are injected with 2.5 cc solutions of plasmid DNA expressing VEGF at eight locations on the lower leg, as sketched in FIG. 18. After each injection, the above described assembly of transducer elements will be centered over the middle of the injection site, with the long axis of the sonication zone parallel to the muscle fibers.

### PART III—PREFERRED EMBODIMENTS OF MULTI BEAM, MULTI PHASE/FREQUENCY EXTERNAL TRANSDUCER SYSTEM 12

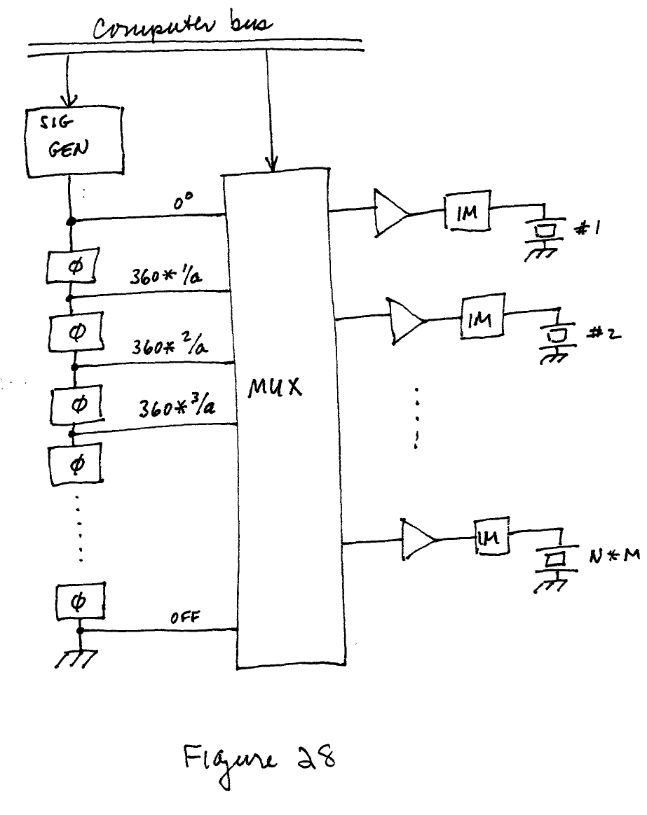

FIG. 19 shows multiple banks of transducers with each bank containing an arc of individual transducers, to sonicate the entire volume of injectate for the purpose of enhancing transfection rates for gene therapy.

In accordance with the present invention, each transducer element of such a multi-transducer assembly may operate at the same time to achieve the same effect, where this operation is either with phase variations among elements or with different emission frequencies.

FIG. 19 depicts a single bank of transducers according to the present invention. The transducers are mounted with a gap between each of the devices to allow for the interleaving of multiple banks, as depicted in FIG. 20. An advantage of this arrangement is that a net uniform dose of ultrasound can be delivered to the tissue volume.

According to the present invention, transducers may be mounted in circular banks to allow for sonication from multiple different directions, an advantage of which is that tissues and bone surrounding the treatment area do not receive excessive exposure of unnecessary ultrasound (and subsequent thermal build up and possible thermal injury). Most preferably, only those tissues at the intersection of the beams from all transducers in a single bank will receive the therapeutic dose of ultrasound. In rabbit studies to date, optimal results have been achieved with a 6 percent duty cycle (DC), however, the present invention is not so limited.

If each bank of devices held “N” transducers, then the effective duty cycle required from each device on a single bank would be 6/N, or more generally DC/N. If the final product contained M banks of transducers, the net duty cycle would then become M* DC/N. Accordingly, for net duty cycles of less than 100 percent, no overlapping operation of transducers would be required, advantageously allowing for a simple switching circuit to drive the multitude of individual transducers. In a preferred aspect, only one transducer would operate at any one time.

In an aspect of the present invention, all transducers on any one bank are operated at the same time (either at the same frequency with phase shifts or at different frequencies).

If all N transducers of a single bank are operated in continuous wave (CW) mode, it is possible to obtain regions of total constructive interference of the beams, regions of total destructive interference, and everything in between. FIG. 21 depicts a simulated CW waveform which results from total constructive interference of six sources, all in phase. The eight CW waveforms follow each other, from top to bottom and from left to right. The numerals on the bottom right side of the graph state the relative frequency and relative phase of the six radiators. Conversely, FIG. 22 depicts a simulated CW waveform which results from total destructive interference of six sources. This interference is created by six identical radiators each 60 degrees out of phase with respect to the next. Note the flat line—no net waveform. Any amplitude waveform in between these two extremes is possible, depending on the phase difference between the signals, as measured at the observation point. FIG. 23, for example, depicts an intermediate amplitude simulated result, in which all six radiators have equal emission, but 30 degree phase differences.

In a first approach, appropriate phases are sequentially selected such that fully constructive interference is created at a multitude of specific sites in the tissue such as to cause fully constructive interference to be resident at these multitude of specific sites in the tissue for the required duty cycle duration. This avoids the problems of CW (or long burst length) emission with hot and cold spots in the beam, wherein some spots may receive sufficient or excessive ultrasound exposure and other spots may receive inadequate exposure.

The physical size of any constructive interference zone will depend on the dimensions of the bank of transducer elements and the proximity of the sample volume to the bank. In a preferred aspect, therefore, the interference zone has a width (parallel to the center of the bank of transducers) on the order of a wavelength and a depth (perpendicular) on the order of 10's of wavelengths. The physical size of the constructive interference zone will also depend on a tolerance for signal drop off. More specifically, an interference zone with signal strength uniformity on the order of 0.5 dB will be substantially smaller than a zone of 3 dB.

In a second approach, the bank of transducers are operated at different frequencies. This second approach also avoids the problems of CW (or long burst length) emission with hot and cold spots in the beam, wherein some spots may receive sufficient or excessive ultrasound exposure and other spots may receive inadequate exposure.

FIGS. 24, 25, 26, and 27 depict simulated waveforms for CW operation where the individual frequencies from six transducers are increments of 1, 2, 4, and 10 percent, respectively. (Phase variations in this case have no meaning—they simply change the time of arrival of peak and null amplitudes.) As can be seen, the net waveforms do not appear significantly different from burst mode operation. Generally, the net waveforms are characterized by N−1 nulls between peak amplitude packets. As can also be seen, the peak 30 amplitude packets have twice the duration as the distance between nulls. The number of cycles between the centers of the peak amplitude packets is proportional to the mean frequency divided by the frequency increment. In accordance with this approach, the only impact of phase is a variation of arrival time of the peak amplitude packet. Consequently, the entire sample volume of tissue within the beams of the transducers which make up the bank will receive the same dose of ultrasound.

A significant advantage of these approaches is that since constructive interference is additive, the individual transducers can be driven at 1/N of their previous drive requirement, but they have to be driven N times longer than previously. Since intensity and power follow as the square of amplitude, the net energy delivered to tissues around the constructive interference zone is advantageously decreased by a factor of N over the amount of exposure from a single element device. This exposure reduction is the same as for that from the previously discussed bank approach.

FIG. 28 depicts a block diagram of a preferred electronic configuration required to drive multiple elements at the same frequency but with variable phase shifters. A computer will define the center frequency of operation. The output frequency will be passed to a series of phase shifters, where the maximum delay is (a−1)/a* 360 degrees, where a is the number of possible phase change increments selected. The different phases are passed to a multiplexer where under computer control the appropriate phase for each element is passed to a power amplifier, an impedance matching circuit, and the subject element. The individual elements my also be passed no signal, equivalent to element OFF.

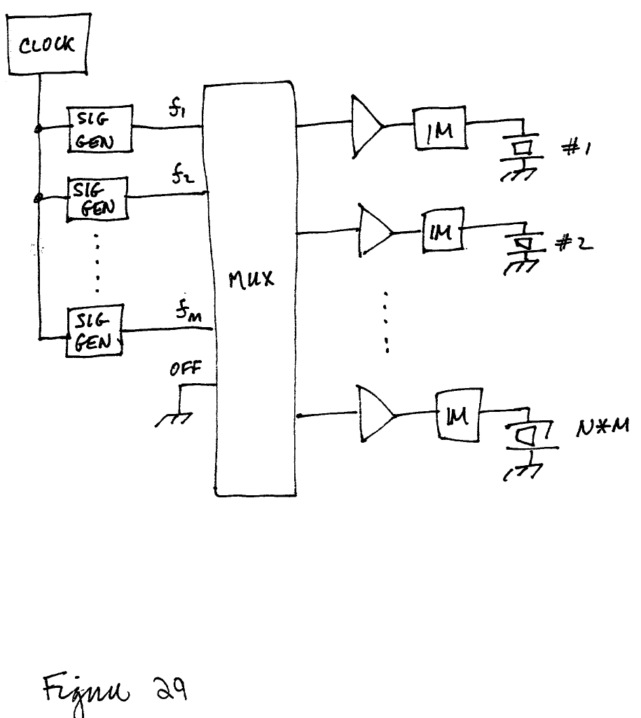

FIG. 29 depicts a block diagram for the multi frequency approach, which comprises a series of signal generators tied to a common clock to generate the specific frequencies. As before, a multiplexer under computer control will preferably assign the appropriate frequencies to all the transducers of not only the bank, but the collections of banks of the entire assembly.

What is claimed is:

1. A combined system for drug delivery and therapeutic ultrasound deliver, comprising:
a catheter having an ultrasound receiver positioned at its distal end.

2. The system of claim 1, further comprising:
an external ultrasound delivery system.

3. The system of claim 2, wherein the ultrasound receiver positioned at the distal end of the catheter assists an operator in directing a beam emitted by the external ultrasound delivery system towards the ultrasound receiver, and thereby, towards the distal end of the catheter.

4. The system of claim 2, wherein,
the external ultrasound delivery system delivers ultrasound of a type to enhance drug delivery uptake.

5. The system of claim 2, wherein,
the external ultrasound delivery system delivers ultrasound of a type to enhance gene delivery transfection.

6. The system of claim 2, wherein,
the external ultrasound delivery system delivers ultrasound of a type to inhibit intimal hyperplasia.

7. The system of claim 2, wherein,
the external ultrasonic ultrasound delivery system delivers ultrasound of a type to enhance a healing response.

8. The system of claim 1, wherein the ultrasound receiver comprises:
a piezoelectric ceramic hydrophone.

9. The system of claim 1, wherein the ultrasound receiver is cylindrical shaped.

10. The system of claim 1, wherein the ultrasound receiver is about 0.07 inches thick, 0.030 inches in outer diameter and about 0.020 inches long.

11. The system of claim 1, further comprising:
a balloon disposed on the distal end of the catheter.

12. The system of claim 2, wherein the external ultrasound delivery system comprises:
a single ultrasound transducer element.

13. The system of claim 2, wherein the external ultrasound delivery system comprises a wide-aperture delivery system which comprises:
a housing;
a large surface area transducer disposed internally at one end of the housing;
a window disposed at an opposite end of the housing; and
a water path between the transducer and the window.

14. The system of claim 13, wherein the transducer is a single circular element.

15. The system of claim 13, wherein the transducer comprises an array of annular elements, one with in another.

16. The system of claim 2, wherein the external ultrasound delivery system comprises:
an array of phased transducers.

17. The system of claim 16, wherein the array of phased transducers is curved, thereby directing ultrasound emissions to a target region of tissue.

18. The system of claim 16, wherein the array of phased transducers is flat, whereby the electronic timing of ultrasonic emission directs the ultrasound beam to a target region in the tissue.

19. The system of claim 17, wherein the array of phased transducers is shaped as an elongated rectangle such that ultrasound emissions are directed to an elongated target region of tissue.

20. The system of claim 16, wherein successive rows of transducers in the array of phased transducers are staggered with respect to one another, such that ultrasound energy is directed to a target region of tissue from several planes.

21. The system of claim 2, wherein the external ultrasound delivery system emits ultrasound with a center frequency of 0.2 to 5.0, and more preferably 0.3 to 3.0, and most preferably about 0.947 MHz.

22. The system of claim 2, wherein the external ultrasound delivery system emits ultrasound with an amplitude of 0.2 to 10.0, and more preferably 0.5 to 5.0, and most preferably about 1.8 MI.

23. The system of claim 2, wherein the external ultrasound delivery system emits ultrasound with a burst mode cycle of 1 to 1000, and more preferably 2 to 5000, and most preferably about 30.

24. The system of claim 2, wherein the external ultrasound delivery system emits ultrasound with a burst mode repetition of 0.1 to 50, and more preferably 1 to 20, and most preferably about 1.893 kHz.

25. The system of claim 2, wherein the external ultrasound delivery system emits ultrasound with a duty cycle of 0.1 to 50, and more preferably 1 to 20, and most preferably about 6.0%.

26. The system of claim 2, wherein the external ultrasound delivery system emits ultrasound with an exposure time of 10 to 1800, and more preferably 30 to 900, and most preferably about 60.0 seconds.

27. The system of claim 2, wherein the external ultrasound delivery system emits ultrasound with a beam width at −6 dB of 2 to 50, and more preferably 5 to 25, and most preferably about 10.0 mm.

28. A method for enhancing drug delivery comprising:
delivering a drug into a target region of tissue with a catheter;
emitting therapeutic ultrasound from an external emitter in a direction generally towards the target region of tissue, the therapeutic ultrasound enhancing drug delivery;
monitoring a signal from an ultrasound receiver positioned on the distal end of the catheter; and
adjusting the direction of the therapeutic ultrasound emitted from the external emitter in response to the strength of the signal from the ultrasound receiver such that the therapeutic ultrasound emitted by the external emitter targets the target region of tissue.

29. A method for enhancing gene transfection, comprising:
delivering a gene into a target region of tissue with a catheter;
emitting therapeutic ultrasound from an external emitter in a direction generally towards the target region of tissue, the therapeutic ultrasound enhancing gene transfection;
monitoring a signal from an ultrasound receiver positioned on the distal end of the catheter; and
adjusting the direction of the therapeutic ultrasound emitted from the external emitter in response to the strength of the signal from the ultrasound receiver such that the therapeutic ultrasound emitted by the external emitter targets the target region of tissue.

30. A method for inhibiting intimal hyperplasia, comprising:
emitting therapeutic ultrasound from an external emitter in a direction generally towards a target region of tissue, the therapeutic ultrasound inhibiting intimal hyperplasia;
monitoring a signal from an ultrasound receiver positioned on the distal end of a catheter disposed in the target regio of tissue; and
adjusting the direction of the therapeutic ultrasound emitted from the external emitter in response to the strength of the signal from the ultrasound receiver such that the therapeutic ultrasound emitted by the external emitter targets the target region of tissue.

31. A system for therapeutic ultrasound, comprising:
a cylindrical surface containing multiple transducer elements on one or more longitudinally parallel banks; and
a water path housing to couple ultrasonic emission to a patient.

32. The system of claim 31, wherein transducer elements on any one bank have intersecting beams.

33. The system of claim 32, wherein the intersecting beams intersect at a common point.

34. The system of claim 31, wherein the transducers on parallel banks are sized and positioned to generate an essentially uniform exposure of ultrasound within the subject.

35. The system of claim 31, wherein individual transducers are energized separately.

36. The system of claim 31, wherein individual transducers of a single bank are energized at the same time.

37. The system of claim 36, wherein the individual transducers are energized at the same frequency but with fixed and variable phases.

38. The system of claim 36, wherein the individual transducers are energized at different frequencies.

39. The system claim 31, wherein individual banks of transducers are interleaved to allow for closer packing of transducer banks for greater uniformity of exposure in the longitudinal direction.

40. A combined system for therapeutic ultrasound, comprising:
a catheter having an ultrasound receiver poisoned at its distal end;
an external therapeutic ultrasound transducer positioned on the skin of the subject;
a control system which provides feedback from the internal catheter sensor to the external therapeutic array.

41. A method of therapeutic ultrasound, comprising:
a catheter having an ultrasound receiver positioned at its distal end;
an external ultrasonic delivery system comprising many phased array elements;
a controller system;
whereby the controller system measures the time of acoustic propagation from each element on the phased array;
whereby further the controller system adjusts the transient times and phases of the phased array elements to direct the ultrasonic beam toward the receiving transducer on the catheter;
whereby further the controller system adjusts the transient times and phases of the phased array elements to sweep the ultrasonic beam in the general proximity of the receiving transducer on the catheter.
